(19) United States
(12) Patent Application Publication
(10) Pub. No.: US 2021/0025883 A1
(43) Pub. Date: Jan. 28, 2021

(54) ACTIVE COMPOSITE MATERIALS FOR MICRO-FLUIDIC AND NANO-FLUIDIC DEVICES

(71) Applicant: VALORBEC SOCIETE EN COMMANDITE, Montreal (CA)

(72) Inventors: MUTHUKUMARAN PACKIRISAMY, PIERREFONDS (CA); JAYAN OZHI KANDATHIL, MONTREAL (CA)

(21) Appl. No.: 15/733,670

(22) PCT Filed: Mar. 28, 2019
(86) PCT No.: PCT/CA2019/000039

§ 371 Date: Sep. 28, 2020

Related U.S. Application Data
(60) Provisional application No. 62/649,405, filed on Mar. 28, 2018.

(51) Int. Cl. G01N 33/543; G01N 27/02; B01L 3/00

(52) U.S. Cl. CPC G01N 33/54373; G01N 27/026; B05D 1/005; B01L 3/502707; B01L 3/502715

(57) ABSTRACT

Microfluidic devices in applications such as biological and chemical sensing provide high sensitivity and exploit minute amounts of samples and reagents in solutions that are low cost, compact, portable and easily distributed to end-users, etc. Polydimethylsiloxane (PDMS) is a common material for the mechanical structure of the body, micro-channels etc. However, its inert nature and biocompatibility mean that active functionality must be added after the formation of the microfluidic structures and structural elements. It would be beneficial to provide designers of microfluidic devices with the ability to implement active elements for the detection of one or more components within a fluid or arising within a fluid from a reaction upstream using a composite comprising a mechanical component, such as the PDMS, with the active material for the active element of the microfluidic device embedded with the PDMS as a composite.

### CROSS-REFERENCE TO RELATED APPLICATIONS

This application claims the benefit of priority as a national phase entry application of World Intellectual Property Office Application PCT/CA2019/000039 filed 28 Mar. 2019 which itself claims the benefit of priority from U.S. Provisional Patent Application 62/649,405 filed 28 Mar. 2018, the entire contents of each being incorporated herein by reference.

### FIELD OF THE INVENTION

This application relates to composite materials and more particularly to composite materials combining an active moiety material with a structural material and their use in supporting novel integration within micro-fluidic and nano-fluidic components and devices.

### BACKGROUND OF THE INVENTION

Microfluidics deals with the behaviour, precise control and manipulation of fluids that are geometrically constrained to a small, typically sub-millimeter, scale. It is a multidisciplinary field involving engineering, physics, chemistry, biochemistry, nanotechnology, and biotechnology, with practical applications in the design of systems in which low volumes of fluids are processed. Since the 1980s micro-fluidics has evolved to use within inkjet printheads, DNA chips, lab-on-a-chip technology, micro-propulsion, and micro-thermal technologies. Micro-fluidic devices may process a single fluid, or they may exploit multiplexing either with automated fluidics, self-driven fluidics, and high-throughput screening. Whilst microfluidics addresses devices that process small volumes, namely femto-liters (fL), pico-liter (pL), nano-liter (nL) and micro-liter (μL), it is also common for devices at the μL level to be referred to a microfluidic whilst devices at the fL, pL and nL level are referred to as nanofluidic. However, in each instance the devices exploit effects of the microdomain, are small, have low power consumption, and within biological and chemical sensing applications also provide high sensitivity, exploit minute amounts of samples and reagents, offer low cost, are portable and easily distributed to end-users, etc.

Typically, within microfluidic devices and systems one or more fluids are moved, mixed, separated or otherwise processed. Numerous applications employ passive fluid control techniques like capillary forces. In some applications, external actuation means are additionally used for a directed transport of the media. Examples are rotary drives applying centrifugal forces for the fluid transport on the passive chips whereas active microfluidics exploit the defined manipulation of the working fluid by active (micro) components such as micropumps or microvalves. Micropumps supply fluids in a continuous manner or are used for dosing. Microvalves determine the flow direction or the mode of movement of pumped liquids. Often processes which are normally carried out in a lab are miniaturised on a single chip (e.g. lab-on-a-chip) in order to enhance efficiency and mobility as well as reducing sample and reagent volumes.

Microfluidic devices are commonly fabricated using polydimethylsiloxane (PDMS) for the mechanical structure of the body, micro-channels etc. PDMS is the most widely used silicon-based organic polymer and is particularly known for its unusual rheological (or flow) properties, being optically clear, and, in general, inert, non-toxic, and non-flammable together with tunable elastomeric properties, gas permeability, low auto fluorescence, nano-scale precision, and easy moldability.

Accordingly, PDMS is an important structural material for the fabrication of microfluidic and nanofluidic devices for various biomedical and industrial applications where its biocompatibility and compatibility with low complexity and low-cost fabrication processes are particularly attractive.

However, its inert nature and biocompatibility mean that all of the active functionality of the microfluidic devices must be added during the fabrication process after the formation of the microfluidic structures and the structural elements. Within the prior art several attempts have been reported within the prior art to enhance the properties of PDMS. These have included reinforcing the PDMS with carbon nanotubes or embedding gold or silver nanoparticles in order to either adjust the mechanical properties of the PDMS or to provide an electrically conductive polymer for support plasmon resonance based devices or electro-mechanical applications. Alternatively, the PDMS has been made porous allowing the introduction of an active material within a hydrogel into the pores.

However, it would be beneficial to provide designers of microfluidic devices with an ability to implement active elements for the detection of one or more components within a fluid or arising within a fluid from a reaction upstream using a composite comprising a mechanical component, such as the PDMS, with the active material for the active element of the microfluidic device embedded with the PDMS as a composite. Such active elements employing a composite from by integrating an active material, reagent, into the structural matrix may support the realization of a variety of applications, including biosensing and sensing applications.

Other aspects and features of the present invention will become apparent to those ordinarily skilled in the art upon review of the following description of specific embodiments of the invention in conjunction with the accompanying figures.

### SUMMARY OF THE INVENTION

This application is directed to composite materials and more particularly to composite materials combining an active moiety material with a structural material and their use in supporting novel integration within micro-fluidic and nano-fluidic components and devices.

In accordance with an embodiment there is provided a device comprising:
a predetermined portion of the device comprising an active material composite comprising a passive material and an active material dispersed within the passive material; wherein
the active material is selected in dependence upon a target analyte to be measured; and
the active material composite further comprises a polymer additive comprising at least one an electrically conductive polymer additive and an electrically non-conductive polymer additive

In accordance with an embodiment there is provided a device comprising:
a predetermined portion of the device comprising an active material composite comprising a passive material and an active material dispersed within the passive material; wherein
the active material is selected in dependence upon a target analyte to be measured;
the active material composite further comprises at least one of microparticles and nanoparticles; and
the microparticles are either solid, hollow, or incorporate a plurality of nanoparticles within a matrix.

In accordance with an embodiment there is provided a microfluidic device comprising:
an active material composite within a predetermined portion of the device, the active material composite comprising a passive material, a first active material supporting an interaction with an analyte and one or more second active material fillers; wherein
the one or more second active fillers are at least one of electrically conducting nanoparticles and electrically non-conducting nanoparticles incorporated with multiple layers of the active material composite to enhance the sensitivity of detecting the analyte interaction by increasing the surface area of first active material exposed to the analyte.

In accordance with an embodiment there is provided a device comprising:
an active material composite within a predetermined portion of the device, the active material composite comprising a passive material, an active material supporting an interaction with an analyte and a filler; wherein
the filler comprises at least one of metallic nanoparticles and electrically conductive nanoparticles; and
an interaction of at least one of a biological analyte and a chemical analyte with the active material is interrogated by electrical impedance spectroscopy.

In accordance with an embodiment there is provided a device comprising:
a predetermined portion of the device comprising an active material composite comprising a passive material and an active material dispersed within the passive material; wherein
the active material is selected in dependence upon a target analyte to be measured.

In accordance with an embodiment there is provided a microfluidic device comprising:
a microfluidic element forming a portion of the microfluidic circuit; wherein
a predetermined portion of the microfluidic element is formed from an active material composite comprising a passive material and an active reagent dispersed within the passive material.

In accordance with an embodiment there is provided a microfluidic circuit comprising:
a microfluidic element forming a portion of the microfluidic circuit; and
a removable element for insertion and removal from the microfluidic element; wherein
the removable element is formed from an active material composite comprising a passive material and an active reagent dispersed within the passive material.

In accordance with an embodiment there is provided a method comprising:
providing a predetermined portion of a device comprising an active material composite comprising a passive material and an active material dispersed within the passive material;
providing an optical source disposed to couple first optical signals into the predetermined portion of the device; and
providing an optical detector disposed to measure second optical signals from the predetermined portion of the device; wherein
the active material is selected in dependence upon a target analyte to be measured.

In accordance with an embodiment there is provided a device comprising:
a predetermined portion of the device comprising an active material composite comprising a passive material and an active material dispersed within the passive material; wherein
the active material is selected in dependence upon a target analyte to be measured.

In accordance with an embodiment there is provided a device comprising:
a predetermined portion of the device comprising an active material composite comprising a passive material and an active material dispersed within the passive material; wherein
the active material is selected in dependence upon a target analyte to be measured; and
the passive material comprises at least one of a substantially non-electrically conductive base elastomer and a substantially non-electrically conductive polymer

In accordance with an embodiment there is provided a device comprising:
a predetermined portion of the device comprising an active material composite comprising a passive material and an active material dispersed within the passive material; wherein
the active material composite comprises at least one of an elastomer and polymer in combination with a plurality of active materials of which the active material is one;
the plurality of active materials are employed at least one of together within a region of the predetermined portion of the device, within multiple regions of the predetermined portion of the device with a different active material in each region, and within multiple regions of the predetermined portion of the device with varying subsets of the plurality of active materials in each region; and
the plurality of active materials are selected in dependence upon one or more analytes to be analysed.

In accordance with an embodiment there is provided a device comprising:
a predetermined portion of the device comprising an active material composite comprising a passive material and an active material dispersed within the passive material; wherein
   the active material is selected in dependence upon a target analyte to be measured; and
   the active material composite further comprises a particulate filler comprising at least one of electrically non-conductive particles and electrically conductive particles.

In accordance with an embodiment there is provided a method comprising:
fabricating a first predetermined portion of a microfluidic circuit from a predetermined material; and
fabricating a second predetermined portion of the microfluidic circuit from a composite material; wherein
the composite material exhibits a change in optical absorption within a predetermined optical wavelength range upon exposure to a predetermined analyte; and
the predetermined material is optically transparent within the predetermined optical wavelength range allowing monitoring of the change in optical absorption through the first predetermined portion of the microfluidic circuit and the second predetermined portion of the microfluidic circuit.

In accordance with an embodiment there is provided a method comprising dissolving ninhydrin within a suitable solvent to form a ninhydrin mixture;
mixing polydimethylsiloxane (PDMS) with a curing agent and base material to form a PDMS mixture;
combining and mixing the ninhydrin mixture and the PDMS mixture;
spin coating the ninhydrin-PDMS mixture to form a thin film; and
processing the thin film to define an active material composite for detecting at least one of ammonia, an amino acid and an NxHy group.

In accordance with an embodiment there is provided a sensing material comprising
a passive structural material; and
an active material exhibiting a variation in a property in dependence upon exposure to a specific analyte or analytes; wherein
the active material and passive structural material are mixed to form a single composite material.

In accordance with an embodiment there is provided a method comprising:
providing a passive structural material;
providing an active material exhibiting a variation in a property in dependence upon exposure to a specific analyte or analytes;
forming a single composite material by mixing the active material and the passive structural material; and
forming either:
   a thin film of the single composite material for removable insertion into a microfluidic circuit as part of a sensor for the specific analyte or analytes; or
   a first monolithically integrated portion of a sensor for the specific analyte or analytes from the single composite material in conjunction with a second monolithically integrated portion of the sensor from a predetermined material.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Various aspects of this disclosure are described, by way of example only, with reference to the attached Figures, wherein:

FIG. 1A depicts an example of a conceptual microfluidic Point-of-Care (POC) device;

FIGS. 1B to 1D depict examples of powering microfluidic devices via chemical reaction, centrifugal force, and capillary action;

FIG. 2A depicts the chemical structure of ninhydrin;

FIG. 2B depicts the reaction of ninhydrin with ammonia;

FIG. 3 depicts a design schematic of the device according to an embodiment;

FIG. 4A depicts photographs of the Ninhydrin-Polydimethylsiloxane (PDMS) composite film before and after exposure to ammonia;

FIG. 4B depicts optical absorbance spectrum of the Ninhydrin-PDMS composite film before and after exposing to the ammonium hydroxide solution;

FIGS. 4C and 4D depict exemplary process flows for forming an active material composite comprising Ninhydrin and PDMS;

FIG. 5 depicts a prototype fabrication assembly of a device according to FIG. 3;

FIG. 6 depicts the prototype device of FIG. 5 during testing and after packaging;

FIG. 7 depicts sensor output voltages measured over time after reaction with ammonium hydroxide solution of various concentrations with a Ninhydrin-PDMS composite film within the prototype device of FIG. 5;

FIG. 8 depicts the sensor output voltage after 5 minutes of reaction with ammonium hydroxide solution of various concentrations with a Ninhydrin-PDMS composite film within the prototype device of FIG. 5;

FIG. 9 depicts the effect of thickness of Ninhydrin-PDMS composite film on the sensor response time with 30 ppm of ammonia

FIG. 10 depicts the slope response of the sensor employing a Ninhydrin-PDMS composite film against the concentration of ammonia;

FIG. 11 depicts the absorbance spectrum of the Ninhydrin-PDMS composite film;

FIG. 12 depicts an exemplary embodiment of an ammonia sensor with electronics hardware to interface with computer

FIG. 13 depicts sensor responses for films before and after exposure to ammonia;

FIGS. 14 and 15 depict embodiments with multiple regions of active material composite between the optical source and optical detector;

FIG. 16 depicts an embodiment with multiple regions of multiple active material composites between optical sources and optical detectors allowing multiple sensors to be disposed within the same microfluidic circuit;

FIG. 17 depicts an embodiment with multiple regions of multiple active material composites between optical sources and optical detectors allowing multiple sensors to be disposed within a microfluidic capillary pump as an example of integration within a microfluidic circuit element;

FIG. 18 depicts examples of microparticles and nanostructures embedded within an active material composite;

FIG. 19 depicts schematically a fluorescent based reagent sensor; and

FIG. 20 depicts an exemplary process flow for fabricating a microfluidic circuit with a PDMS based active material composite through lithography, deposition and lift-off.

### DETAILED DESCRIPTION

This application is directed to composite materials and more particularly to composite materials combining an active moiety material with a structural material and their use in supporting novel integration within micro-fluidic and nano-fluidic components and devices.

The ensuing description provides representative embodiment(s) only, and is not intended to limit the scope, applicability or configuration of the disclosure. Rather, the ensuing description of the embodiment(s) will provide those skilled in the art with an enabling description for implementing an embodiment or embodiments of the invention. It being understood that various changes can be made in the function and arrangement of elements without departing from the spirit and scope as set forth in the appended claims. Accordingly, an embodiment is an example or implementation of the inventions and not the sole implementation. Various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention can also be implemented in a single embodiment or any combination of embodiments.

Reference in the specification to “one embodiment”, “an embodiment”, “some embodiments” or “other embodiments” means that a particular feature, structure, or characteristic described in connection with the embodiments is included in at least one embodiment, but not necessarily all embodiments, of the inventions. The phraseology and terminology employed herein is not to be construed as limiting but is for descriptive purpose only. It is to be understood that where the claims or specification refer to “a” or “an” element, such reference is not to be construed as there being only one of that element. It is to be understood that where the specification states that a component feature, structure, or characteristic “may”, “might”, “can” or “could” be included, that particular component, feature, structure, or characteristic is not required to be included.

Reference to terms such as “left”, “right”, “top”, “bottom”, “front” and “back” are intended for use in respect to the orientation of the particular feature, structure, or element within the figures depicting embodiments of the invention. Such directional terminology with respect to the actual use of a device has no specific meaning as the device can be employed in a multiplicity of orientations by the user or users.

Reference to terms “including”, “comprising”, “consisting” and grammatical variants thereof do not preclude the addition of one or more components, features, steps, integers or groups thereof and that the terms are not to be construed as specifying components, features, steps or integers. Likewise, the phrase “consisting essentially of”, and grammatical variants thereof, when used herein is not to be construed as excluding additional components, steps, features integers or groups thereof but rather that the additional features, integers, steps, components or groups thereof do not materially alter the basic and novel characteristics of the claimed composition, device or method. If the specification or claims refer to “an additional” element, that does not preclude there being more than one of the additional element.

An “analyte” as used herein and throughout this disclosure may refer to, but is not limited to, a component (in clinical chemistry), chemical species, substance or chemical constituent that is of interest in an analytical procedure.

A “moiety” as used herein and throughout this disclosure may refer to, but is not limited to, a part of a molecule forming a functional group. A moiety or functional group participates in similar chemical reactions in most molecules that contain it.

A “microstructure” as used herein and throughout this disclosure may refer to, but is not limited to, the small scale structure of a material.

A “nanostructure” as used herein and throughout this disclosure may refer to, but is not limited to, a structure of intermediate size between microscopic and molecular structures. Nanostructural detail is microstructure at nanoscale.

A “microparticle” as used herein and throughout this disclosure may refer to, but is not limited to, particles between 0.1 and 100 micrometres (μm) in size.

A “nanoparticle” as used herein and throughout this disclosure may refer to, but is not limited to, particles between 1 and 100 nanometres (nm) in size with a surrounding interfacial layer. The interfacial layer is an integral part of nanoscale matter, fundamentally affecting all of its properties. The interfacial layer typically consists of ions, inorganic and organic molecules.

“LIGA” as used herein and throughout this disclosure may refer to, but is not limited to, a fabrication technology for the formation of high aspect ratio microstructures. Derived from the German acronym Lithographie, Galvanoformung, Abformung the process comprises three main processing steps of Lithography, Electroplating, and Molding. Common LIGA fabrication technologies are X-ray LIGA and UV LIGA.

An “optical source” as used herein and throughout this disclosure may refer to, but is not limited to, a light emitting diode (LED), a laser diode (LD), a diode pumped solid state source (DPSS), and a dye laser. Dye lasers may be formed within the microfluidic circuit as these generally employ liquid dyes. An optical source may be narrow linewidth, e.g. distributed feedback laser (DFB); narrowband, e.g. Fabry-Perot laser; wideband, e.g. LED; filtered; unfiltered; fixed wavelength; tunable wavelength; continuous wave; pulsed; and modulated.

An “optical detector” as used herein and throughout this disclosure may refer to, but is not limited to, a photodiode, a phototransistor (or light dependent resistor), an avalanche photodetector (APD), a reverse biased LED, and a quantum dot photodetector.

A “lock-in amplifier” as used herein and throughout this disclosure may refer to, but is not limited to, an amplifier capable of extracting a signal with a known carrier wave from an extremely noisy environment. Generally comprising a homodyne detector followed by low-pass filter, which may be static where a fixed carrier wave frequency is employed or adjustable in cut-off frequency and filter order in other instances where the carrier frequency modulating the signal being detected may be referenced from an external modulation source or generated by the lock-in amplifier itself. Analog lock-in amplifiers exploit analog frequency mixers and RC filters for the demodulation whereas digital lock-in amplifiers implement and perform both steps implemented by fast digital signal processing, for example, on a Field Programmable Gate Array.

“SU8” as used herein and throughout this disclosure refers to an epoxy-based negative photoresist. SU8 being composed of Bisphenol A Novolac epoxy dissolved in an organic solvent (for example gamma-butyrolactone (GBL) or cyclopentanone) and a photoacid generator (for example, up to 10 wt % of mixed triarylsulfonium/hexafluoroantimonate salt).

### A: MICRO-FLUIDIC DEVICES

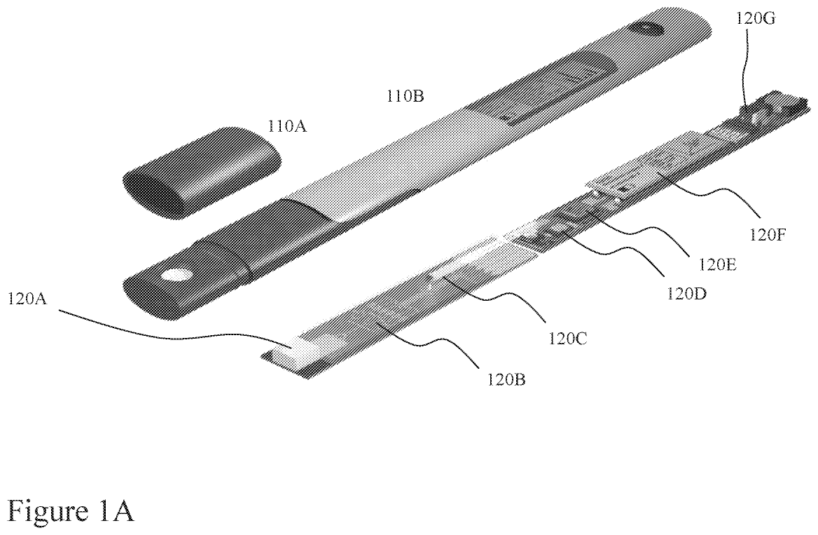

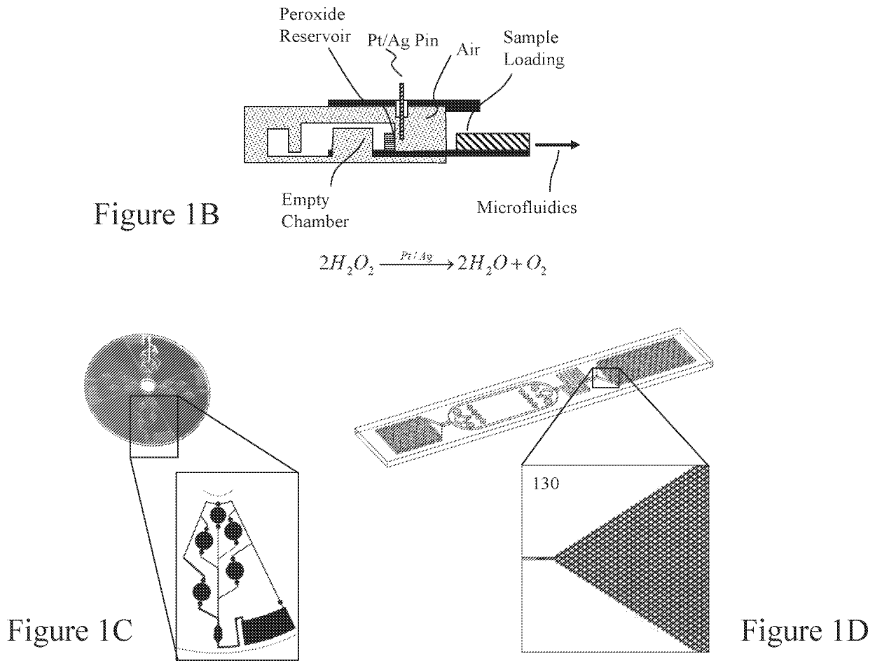

Referring to FIG. 1A there is depicted an example of a conceptual microfluidic Point-of-Care (POC) device after Gervais et al in “Microfluidic Chips for Point-of-Care Immunodiagnostics” (Advanced Materials, Vol. 23(24), pp. H151-H176, hereinafter Gervais1) wherein a POC tester comprising a body 110A and cover 110B allows a user to perform a measurement or measurements based upon the provisioning of a sample and its initial processing in sample processor 120A. Sample processor 120A for example performing cell separation, cell pre-treatment, pre-concentration or amplification prior to the sample entering a microfluidic chip 120B which is optically interrogated with optical head 120C coupled to opto-electronic circuit 120D. The optical signal is then processed by signal processing electronics 120E which may for example include signal processing, signal encryption, wireless interface, wired interface and logic. The results are presented to the user on display 120F. The body 110A of the POC tester may also include ancillary electronics 120G, such as power supply, USB connector and antenna for example. Accordingly, the microfluidic chip 120B may within embodiments of the invention exploit one or more active material structural composites either as the main structure of the microfluidic chip 120B or within selective areas such as described below.

Microfluidic devices may exploit different “actuation” mechanisms for the transport of the one or more fluids within the microfluidic device. These are divided into actuation mechanisms that are internal (or integrated) to the microfluidic device or external to the microfluidic device within the overall microfluidic system. Those external to the microfluidic device are typically one of the multiple designs of fluidic pump known within the prior art including, but not limited to, positive displacement and centrifugal type such as rotary-type positive displacement, reciprocating-type positive displacement, linear-type positive displacement, impulse pumps, gravity pumps, velocity pumps, etc.

Of the many internal—integrated microfluidic systems developed for POC these include, but are not limited to:
electrokinetically driven microfluidics, see for example “Electrokinetics in Microfluidics, Volume 2 (Interface Science and Technology)” (Elsevier, ISBN-13: 978-0120884445), and whilst powerful require high voltages making for complex systems to operate them;
pneumatically actuated systems, see for example Braschler et al in “A simple Pneumatic Setup for Driving Microfluidics” (Lab on a Chip, Vol. 7, pp. 420-422), but necessitate large valves;
chemical reaction driven microfluidic pumps such as depicted in FIG. 1B, see for example Qin et al in “Self-Powered Microfluidic Chips for Multiplexed Protein Assays from Whole Blood” (Lab on a Chip, Vol. 9(14), pp. 2016-2020). Within the example shown a sample loaded into the microfluidic assembly is driven through the microfluidic channels by pressure arising from the generation of oxygen as a result of a Pt/Ag catalytic breakdown of hydrogen peroxide into water and oxygen within another part of the microfluidic device which then drives the sample through the microfluidic channels;
centrifugally driven microfluidics such as depicted in FIG. 1C, so called “Lab-on-a-CD” requiring tailor-made spinning systems, wherein centrifugal force generates the fluid flow, see for example Lai et al in “Design of a Compact Disk-like Microfluidic Platform for Enzyme-Linked Immunosorbent Assay” (Anal. Chem., Vol. 76, pp. 1832-1837) providing a five-step flow sequence; and
capillary microfluidics constitute the most successful technology to date for assays, see for example Betancur et al in “Integrated Lateral Flow Device for Flow Control with Blood Separation and Biosensing” (Micromachines, Vol. 8(12), pp. 367) wherein a capillary micropump 130 provides “suction” through capillary action to draw the fluid(s) through the microfluidic device.

As will become evident from the discussion below embodiments of the invention are compatible with any microfluidic devices exploiting these microfluidic actuators exploiting one or more active material structural composites either as the main structure of the microfluidic device or within selective areas such as described below.

Further, one or more active material structural composites may be employed either as the main structure of the microfluidic device or within selective areas of the microfluidic component. Further, due to the nature of composites according to embodiments of the invention they may be employed within powered and self-powered, self-regulated microfluidic devices and may be employed in forming one or more capillary elements within the microfluidic circuit including, but not limited to:
microchannels which are closed channels employing hydrophilic or plasma treated conduit surfaces;
serpentine flow resistors which regulate flow rate over a desired region;
delay lines which are typically binary hierarchy microchannels combining flows from a sample collector for example;
vents which are openings within the microfluidic circuit connected to air allowing air to be vented from a closed channel as a fluid fills part of the closed channel;
capillary pumps, such as depicted in FIG. 1B, which are microstructured reservoirs, typically with hydrophilic posts, to generate capillary pressure over a desired region without a significant resistance;
capillary retention valves wherein localized channel cross-section reduction creates a high capillary pressure thereby pinning the fluid after a capillary has been drained;
capillary trigger valves (CTVs) which are formed as a channel intersects (crosses) a main channel wherein the fluid in the cross-channel is retained for a period of time until its release is triggered by another fluid in the main channel;
flow routers;
sequential programmable capillary retention valves employing capillary retention valves;
positive pressure programmable retention burst valves employing retention burst valves such as low pressure and high pressure;
programmable capillary trigger valve employing capillary trigger valve;
programmable capillary pumps employing symmetric and asymmetric capillary pumps such as low pressure and high pressure pumps.

### B: ACTIVE MATERIAL COMPOSITE COMPOSITION OPTIONS

B1: Host Material

Within the embodiments described and depicted in respect of FIGS. 1 to 20 the composite is described and depicted with respect to forming a composite comprising an active material with polydimethylsiloxane (PDMS) as the host material. However, alternatively other elastomers and/or polymers may be employed either discretely or in combination with one or more other elastomers and/or other polymers provided that the material(s) is(are) compatible with the fluid(s) being processed and/or handled by the microfluidic device and/or microfluidic circuit for the projected active lifetime of the microfluidic device. Accordingly, these other materials may provide host materials for the active material. Optionally, a microfluidic device and/or microfluidic circuit may exploit, but not be limited to:
the active material composite solely;
one or more regions with the active material composite in combination with one or more base matrices defined by one or more sol-gel processes;
one or more regions with multiple active material composites with one or more base matrices defined by one or more sol-gel processes;
one or more regions with the active material composite in combination with one or more base elastomers; and
one or more regions with multiple active material composites with one or more base elastomers.

Optionally an elastomer and/or polymer according to an embodiment may comprise one or more of polydimethylsiloxane (PDMS), poly(methyl methacrylate) (PMMA), polystyrene (PS), polyvinyl chloride (PVC), polytetrafluoroethylenes (PTFE), cellulose derivatives such as ethyl cellulose, SU8, and cyclic olefin copolymer (COC)/cyclic olefin polymer (COP).

Optionally, the PDMS may according to embodiments of the invention comprise polydimethylsiloxane (PDMS) and a polysiloxane sealant formulation comprising from 85.0-100 wt % of hydroxy-terminated polydimethylsiloxane, from 7.0-13.0 wt % of amorphous fumed silica, and from 1.0-5.0 wt % of methyltriacetoxysilane. Optionally, the PDMS may be employed in conjunction with a curing agent suitable for curing PDMS.

Optionally, embodiments of the invention may exploit an initial base elastomer and/or polymer, which may for example be a substantially non-electrically conductive, silicone-based base elastomer and/or polymer, in combination with one or more active materials.

Optionally, embodiments of the invention may exploit an initial base elastomer and/or polymer, which may for example be a substantially non-electrically conductive, base elastomer and/or polymer, in combination with one or more active materials.

Optionally, embodiments of the invention may exploit an initial base elastomer and/or polymer in combination with one or more active materials wherein the one or more active materials are at least one of employed together within a region of a microfluidics device, within multiple regions of the microfluidic device with a different active material in each region, and within multiple regions of the microfluidic device with varying subsets of the one or more active materials in each region.

Optionally, the active material composition according to embodiments of the invention may also employ a particulate filler comprising at least one of electrically non-conductive particles and electrically conductive particles in combination with at least one of the active material composite and a structural material of the microfluidics device.

Optionally, the active material composition according to embodiments of the invention may also employ a polymer additive comprising at least one an electrically conductive polymer additive and an electrically non-conductive polymer additive in combination with at least one of the active material composite and a structural material of the microfluidics device.

Optionally, one or more regions of the microfluidics device comprising at least one electrically conductive polymer additive and an electrically non-conductive polymer additive may, within embodiments of invention, be disposed within regions of the microfluidics device to provide modified structural properties. Said regions may be where the active material composite is employed, adjacent to other regions where the active material composite is employed, or in other regions.

An electrically conductive polymer additive according to embodiments of the invention may comprise one or more of: ethylenedioxythiophene (EDOT); poly(3,4-ethylenedioxythiophene) (PEDOT); PEDOT doped with poly(styrenesulfonate) (PEDOT/PSS); polyaniline; poly(pyrrole); poly(acetylene); poly(thiophene); poly(p-phenylene sulfide); poly(para-phenylene vinylene) (PPV); polyindole; polypyrene; polycarbazole; polyazulene; polyazepine; polynaphthalene; other conjugated polymers and derivatives of these materials.

The particulate filler according to embodiments of the invention may comprise of one or more of metal-based microparticles, metal-based nanoparticles, carbon-based microparticles, carbon-based nanoparticles, silica nanoparticles, alumina nanoparticles, and zirconia nanoparticles.

Carbon-based microparticles and nanoparticles according to embodiments of the invention may comprise one or more of single walled carbon nanotubes, multi-walled carbon nanotubes, carbon nanorods, graphene, graphite and fullerene. Said carbon-based microparticles and nanoparticles may within embodiments of invention be disposed within regions of the microfluidics device to provide modified structural properties. Said regions may be where the active material composite is employed, adjacent to other regions where the active material composite is employed, or in other regions.

Metal based microparticles and nanoparticles according to embodiments of the invention may comprise one or more silver, gold, platinum, copper, nickel, aluminum, zinc, molybdenum, cadmium, scandium, titanium, vanadium, chromium, manganese, iron, cobalt, yttrium, zirconium, niobium, tantalum, tungsten, lead, indium tin oxide, terfenol-D, manganin and constantan. Said metal-based microparticles and nanoparticles may within embodiments of invention be disposed within regions of the microfluidics device to provide modified structural properties. Said regions may be where the active material composite is employed, adjacent to other regions where the active material composite is employed, or in other regions.

The nanoparticles composite with analytes can also change the electrical impedance of the regions comprising the active material composite. As a result, bioimpedance would vary with the sensing and interaction with analyte which can be measured through electrical impedance and/or electrical impedance spectroscopy.

B2: Active Material Options

Within the embodiments described and depicted in respect of FIGS. 1 to 20 the composite is described and depicted with respect to forming a composite comprising ninhydrin as the active material with polydimethylsiloxane (PDMS) as the host material. However, other reagent mediated sensors exploiting different active materials may be employed including, but not limited to those exploiting:
Optical Absorption, either increasing or decreasing with analyte reaction with the reagent, i.e. active material;
Optical Luminescence, such as fluorescent and phosphorescence as described below in Section G4 and FIG. 19;
Optical Luminescence Lifetime based sensing; and
Electrochemical.
Electrical such as bioimpedance

Active materials in each instance have properties, optical or electrical for example, that are affected by various analytes. The analytes detected may be primary analytes, e.g. directly measured within the sample being analysed, or be second analytes, e.g. analytes established based upon chemical reaction of a primary analyte wherein the second analyte is measured to derive the presence and/or concentration etc. of the primary analyte.

### C: EXAMPLE OF ACTIVE MATERIAL COMPOSITE AMMONIA SENSOR

C1: Background

Ammonia is present almost everywhere in the atmosphere at a low concentration, in the range of sub-parts per billion (ppb) levels. Ammonia is a compound present in environmental samples such as aquaculture effluents, industrial wastewaters, plants and soil, and in pharmaceutical formulations for example. The majority of the ammonia present in atmosphere arises from human activities among which agricultural activities such as livestock and fertilizers are the most prominent sources of ammonia. Other sources of ammonia include on-road vehicles, industrial chemical plants and silver chemicals. However, extensive exposure to ammonia is harmful to both animals and humans. Ammonia at a level of 24-50 parts per million (ppm) can irritate the nose and the throat whilst at a moderate and higher exposure of typically above few hundreds of ppm of exposure to only a few minutes can cause severe irritation in the respiratory tract, spasms, and rapid suffocation. Accordingly, the acceptable threshold limit value (TLV) for ammonia gas with an exposure longer than 8 hours is around 25 ppm: and short-term exposure is 35 ppm for 15 minutes in order to serious health issues.

Accordingly, it is important to be able to detect ammonia in air at low concentrations (ppm level) for controlling pollution and in industrial processes such as food technology, fertilizers, and: especially, in environments where refrigeration processes are carried out. Several methods have been reported within the prior art for the determination of ammonia including spectrophotometry, solid-phase extraction, diffuse reflectance spectroscopy, electrochemical methods, ion-chromatography, spectrofluorimetry, capillary coulometric titration using the hypochlorite luminol chemiluminescence reaction, potentiometry with a differential system or indirect methods: for example, by amperometric detection of ammonium ions. Usually, the spectrophotometric method is used for the detection of ammonia and it is based on the adaptation of classical methods such as the Nessler or Berthelot reactions.

The major application areas of ammonia sensors are gas sensing and analysis in automotive industry, chemical industry and biomedical industry. Ammonia sensors having high sensitivity can be used as disease diagnostic tool as the ammonia is natural body product and its detection arid measurement can precisely predict the health conditions of various internal organs such a kidney and liver where breath ammonia detection can be used as fast diagnostic technique for patients having a kidney disorder or a stomach ulcer.

Within the prior art ammonia sensors have been reported and are generally categorized into five main types, each with its own advantages and disadvantages. These being metal oxide semiconductor devices, catalytic ammonia sensor, conducting polymer gas sensors, optical gas sensing, and indirect gas analyzers. Many of the classical analytical techniques used for the detection of ammonia suffer from drawbacks and are not suitable for the miniaturization into convenient POC sensors.

At present there is increased demand for miniaturized ammonia sensors due to several benefits such as faster analysis, low cost, easy integration with PEDs, FEDs, and other instruments and easy integration with miniaturized air vehicles such as drones in order to map the presence of ammonia, for environmental or industrial safety monitoring etc.

Accordingly, considering the prior art then several sensing principles for measuring ammonia in air have been reported but typically exploit infrared gas analyzers which are large and expensive, and not suitable for miniaturization and integration in a chip either as a discrete ammonia sensor or in combination with sensors for other materials and/or environment. Methods, more suitable for miniaturization are those based on the semiconductor properties of metal thin films (such as tin oxide or molybdenum oxide) or conducting polymer film gas sensors. However, their detection limits are not low enough and the selectivity of the methods is poor for many applications. In addition, the sensors lifetime has been found limited together with restrictions concerning their reproducibility, stability, sensitivity, and selectivity.

Other methods are based on the absorption of ammonia into a liquid and the subsequent detection of the ammonium ions by using an electrolyte conductivity detector. However, these techniques are bulky, measure cumulative ammonia and whilst electrochemical methods are sensitive and selective the instruments are quite expensive, and the presence of an experienced operator is either required for fast turnaround or samples must be sent for analysis.

More recently approaches have been reported for the fabrication of optical gaseous ammonia sensors which utilize the reaction of ammonia vapor with either a pH-dependent dye material or a pH-sensitive film which undergoes a suitable color change or an absorption change In general, these sensing mechanisms are based on monitoring the absorption or fluorescence characteristics of indicator dyes/sensing films entrapped within a membrane, deposited onto a wave guiding substrate or an optical fiber as substituted cladding. The targeted ammonia molecules interact with the immobilized indicator, resulting in changes in their absorbance or emission spectra, which are monitored using a proper detector module, via an optical fiber or optical waveguide.

C2: Concept for Optical Detection of Ammonia Using Ninhydrin-PDMS Composite

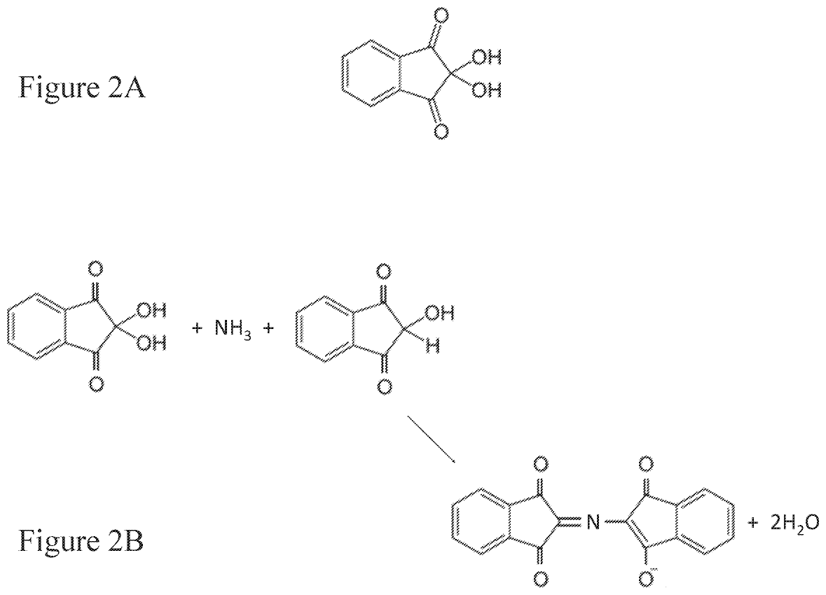

In order to demonstrate the active material polymer composite according to an embodiment the inventors established a composite comprising 2,2-dihydroxyindane-1,3-dione (known as ninhydrin) for the detection of ammonia within polydimethylsiloxane (hereinafter referred to as Ninhydrin-PDMS composite). Referring to FIG. 2A the structure of ninhydrin is depicted whilst FIG. 2B depicts the overall reaction mechanism of ammonia with ninhydrin. The reaction generates the ninhydrin chromophore (2-(1,3-dioxoindan-2-yl)iminoindane-1,3-dione) wherein the amine is condensed with a molecule of ninhydrin to give a Schiff base, Ruhemann's Purple, via a reversible reaction. Only ammonia and primary amines can achieve this as an alpha hydrogen is required to form the Schiff base. Accordingly, amines bound to tertiary carbons do not react and cannot be detected, e.g. secondary amines.

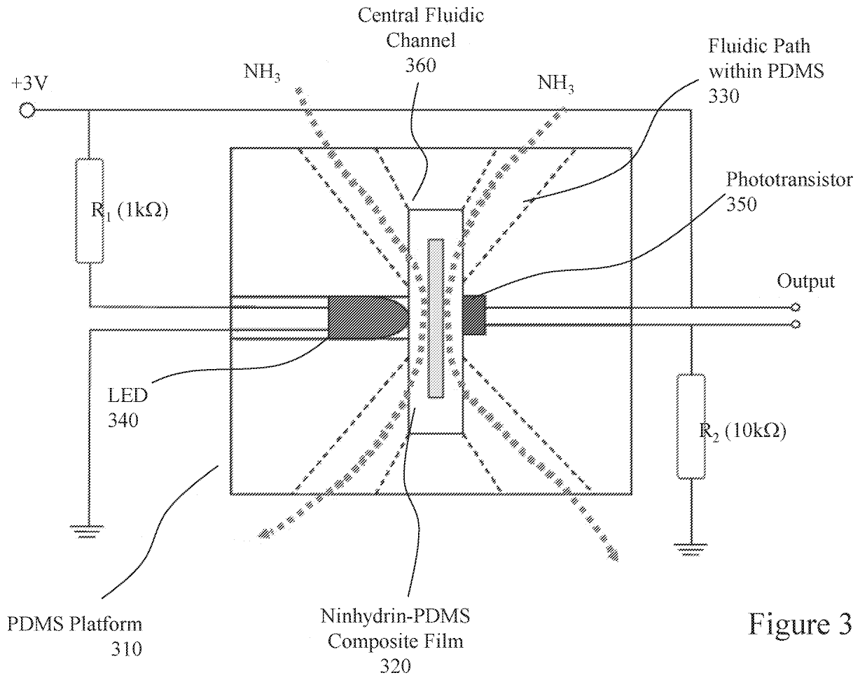

Now referring to FIG. 3 there is depicted an exemplary microfluidic device for the detection of ammonia based upon the chemical process depicted in FIG. 2B. Accordingly, as depicted A PDMS platform 310 contains a slot within an overall 4 mm wide central channel 360 for fixing a Ninhydrin-PDMS composite film 320 within such that it is within the optical path between the LED 340 and Phototransistor 350. Two fluidic paths 330, similarly of width 4 mm, were provided to flow the ammonia gas to the Ninhydrin-PDMS composite film 320 sensing element.

The device is powered by 3V DC power supply wherein the LED is biased via a first resistor R1 (1 kΩ) and the phototransistor 350 via a second resistor R2 (10 kΩ). The voltage across the photoresistor 350 was measured in order to quantify the amount of ammonia reacting with film and accordingly quantifies the concentration of ammonia.

C3: Fabrication of the Ninhydrin-PDMS Composite Film

Within the initial proof of concept device depicted within FIG. 3 the active material composite, in this example a Ninhydrin-PDMS composite was implemented as a film which was inserted within a slot formed within a PDMS microfluidic device. However, as evident from FIGS. 14 to ZZZ the Ninhydrin-PDMS composite may be formed as part of the microfluidic device by other methods including, but not limited to, casting, spinning, and coating. Accordingly, a sensing platform of an active material polymer composite, e.g. a thin film, containing ultrafine particles of the active material, e.g. ninhydrin, within the polymer, e.g. PDMS, is provided.

Within the initial prototype devices sensing films of 100-1,000 μm were fabricated although other thicknesses may be exploited either per element or in overall combination within the optical path from the optical source, e.g. LED, to optical detector.

Synthesis of the ninhydrin-PDMS composite started by mixing the PDMS and a curing agent, e.g. at 10:1 wt %. Separately, the ninhydrin was dissolved in ethanol, e.g. 0.5 g in 5 ml, and stirred until all the ninhydrin had dissolved. The ninhydrin solution was added to the PDMS mixture and stirred for about 5 minutes. Immediately after adding the ninhydrin solution, the PDMS mixture appears to a low viscosity, then slowly, the PDMS mixture becomes viscous. At this point the mixture was degasified to remove any air bubbles. The film was fabricated by spinning, e.g. 300 rpm for 30 seconds, onto a carrier wafer, e.g. a silicon wafer. The silicon wafer was silanized prior to coating to promote the easy removal of the Ninhydrin-PDMS composite film. After spinning the Ninhydrin-PDMS composite film was baked at 85° C. for 2 hours and then peeled off from the wafer.

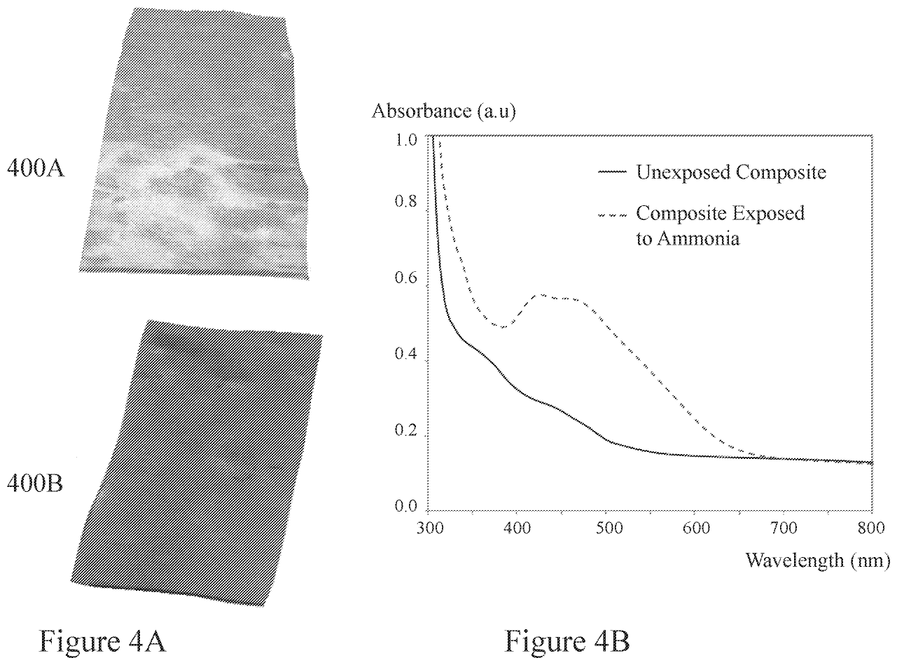

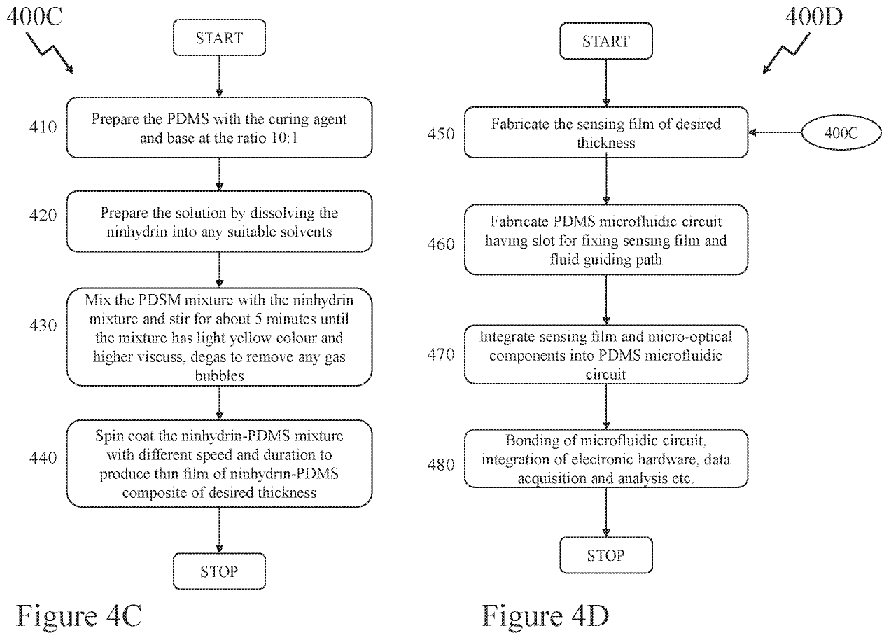

First and second images 400A and 400B in FIG. 4A depict photographs of the Ninhydrin-PDMS composite film before and after exposure to ammonium hydroxide solution. The optical absorbance spectrum of the Ninhydrin-PDMS composite film before and after exposure to the ammonium hydroxide solution is depicted in FIG. 4B. As evident from the upper dashed curve after exposure to the ammonium hydroxide solution there is a high absorbance window with the wavelength range of 420 nm and 480 nm in the blue-purple region of the visible spectrum. Hence an LED with an emission band of 465-475 nm was used in the device InGaN, GaN and InGaN based semiconductor lasers and LEDs operating in the wavelength ranges 445 nm-465 nm and 380 nm-450 nm may be employed, preferably centred towards the peak absorbance although techniques for enhanced signal detection such as modulating the source and employing a lock-in amplifier etc. may be employed.

Now referring to FIG. 4C there is depicted a process flow 400C for this exemplary Ninhydrin-PDMS Composite film according to an embodiment. As depicted the process comprises first to fourth steps 410 to 440 respectively comprising:
First step 410 comprising preparing the PDMS with the curing agent and base at the ratio 10:1;
Second step 420 comprising preparing the solution by dissolving the ninhydrin into any suitable solvents;
Third step 430 comprising mixing the PDSM mixture with the ninhydrin mixture and stir for about 5 minutes until the mixture has light yellow colour and higher viscosity, degas to remove any gas bubbles; and
Fourth step 440 comprising spin coating the ninhydrin-PDMS mixture with different speed and duration to produce thin film of ninhydrin-PDMS composite of desired thickness.

Now referring to FIG. 4D there is depicted a process flow 400D for providing the exemplary microfluidic device with the Ninhydrin-PDMS Composite film according to an embodiment. As depicted the process comprises first to fourth steps 450 to 480 respectively comprising:
First step 450 comprising spin coating the ninhydrin-PDMS mixture with different speed and duration to produce thin film of ninhydrin-PDMS composite of desired thickness (basically fourth step 400D of process flow 400D in FIG. 4C;
Second step 460 comprising fabricating PDMS microfluidic circuit having slot for fixing sensing film and fluid guiding path;
Third step 470 comprising integrating the sensing film and micro-optical components into PDMS microfluidic circuit; and
Fourth step 480 comprising bonding of microfluidic circuit, integration of electronic hardware, data acquisition and analysis etc.

C4: Fabrication Assembly of Device for Ninhydrin-PDMS Composite Film

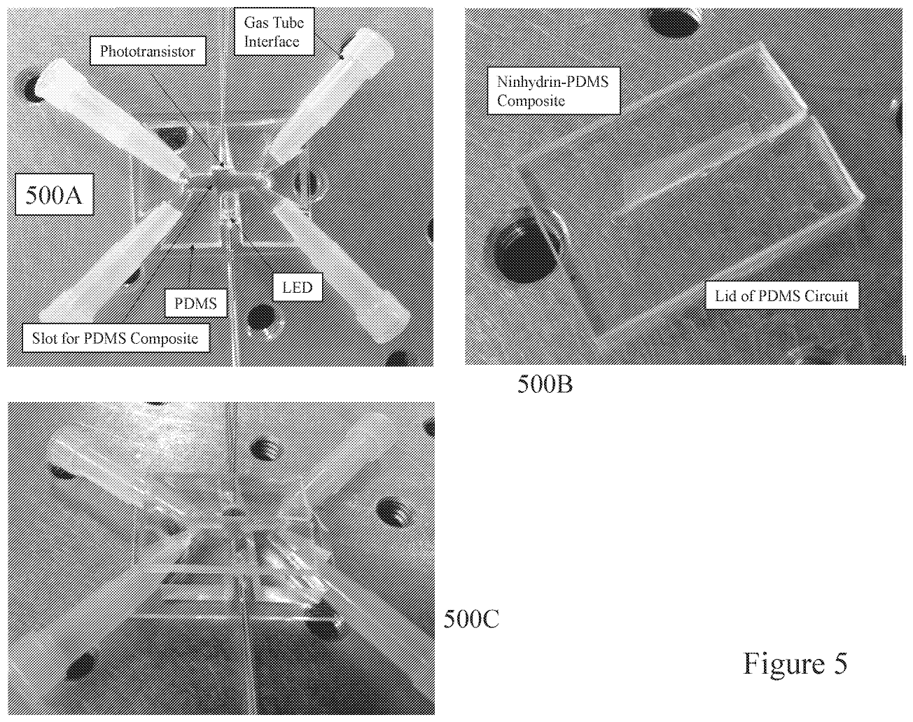

Now referring to FIG. 5 in first to third images 500A to 500C respectively there are depicted the fabricated assembly of the device comprising, respectively:
First image 500A depicting the PDMS platform with channel for gas and fluidic tube for guiding gas, LED and photoresistor;
Second image 500B depicting the top lid of the device with ninhydrin-PDMS composite; and
Third image 500C depicting the assembly after bonding the top lid with the PDMS platform.

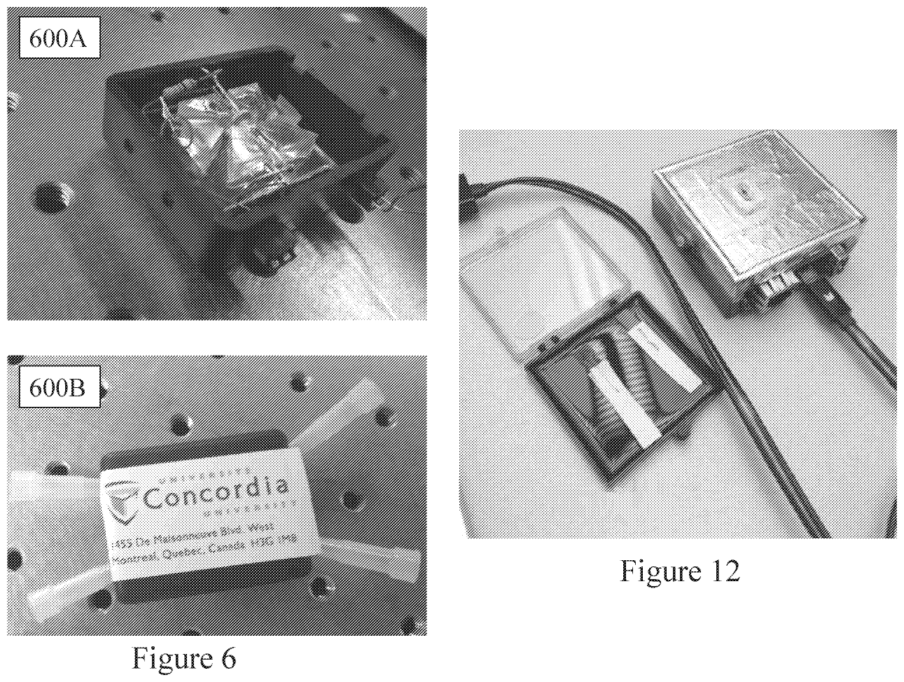

Accordingly, a PDMS platform containing the channel and slots for guiding the gas were bonded onto a glass substrate using oxygen plasma bonding. The plastic horn shaped tubes, which were used as the guiding assembly for the gas to the sensing film, were then fixed in the slots at the input and output points. The LED and photoresistor were then integrated into PDMS in their respective locations opposing one another with the central channel and slot between them. The Ninhydrin-PDMS Composite film was cut to 3×6 mm and affixed on a flat PDMS substrate with a groove in this substrate which would become the top lid of the PDMS platform. The lid was then placed over the PDMS platform with the Ninhydrin-PDMS Composite film in the slot and the lid attached. FIG. 6 depicts first and second images 600A and 600B of the device taken during subsequent stages of the development during testing and after packaging respectively.

C5: Testing of Prototype Ninhydrin-PDMS Composite Film Based Microfluidic Device

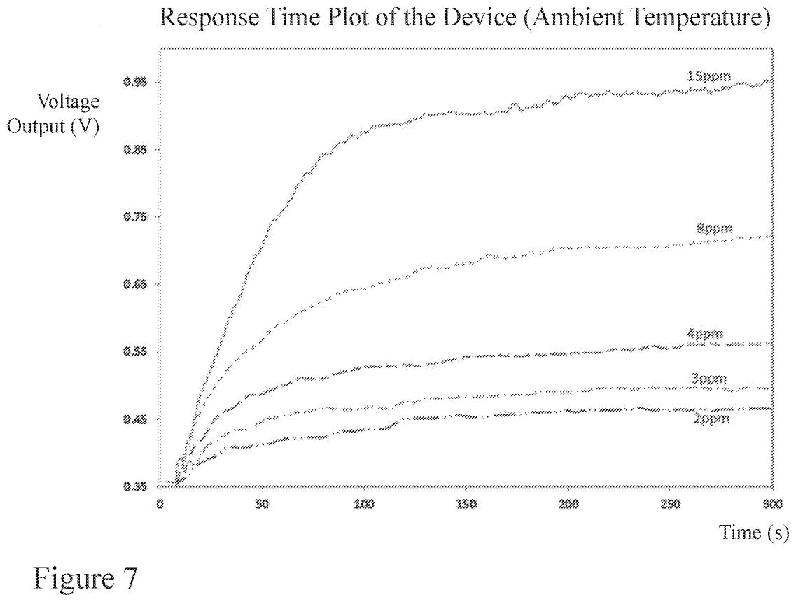

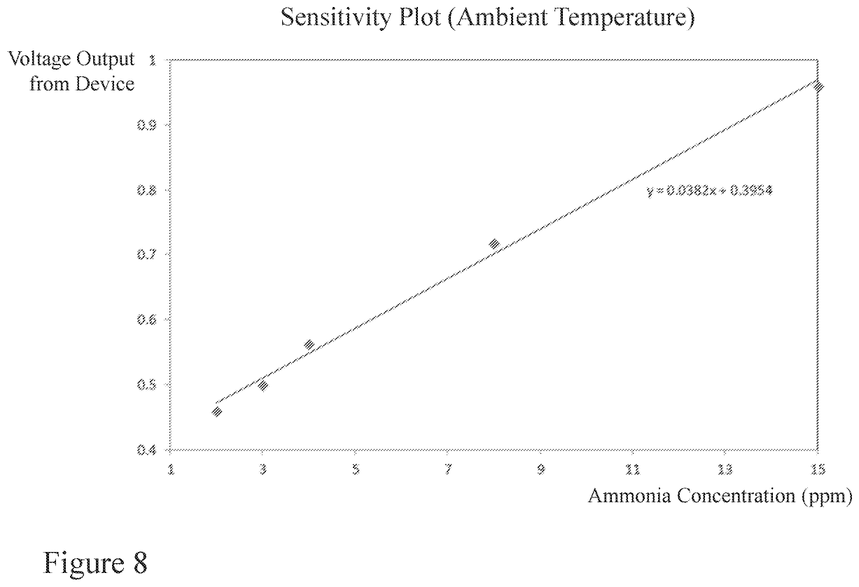

Once assembled the microfluidic device with Ninhydrin-PDMS Composite film then FIG. 7 shows the response of the device for various concentrations of the ammonium hydroxide solution. The concentration of ammonia was estimated and given in FIG. 7. When the solution was passed through the channel, the Ninhydrin-PDMS composite film turned to purple. The time response in FIG. 7 also shows the response time of the sensor. The graph shows that the sensor response has two well defined regions as shown in FIG. 7, the transient region and the saturation region. This indicates that the sensor film slowly reacts with the ninhydrin and eventually gets saturated. It is noticed that the transient and saturation responses are strongly related with the concentration of ammonia and hence, both the responses can be used for quantifying the concentration of ammonia. When the concentration of ammonia was 15 ppm, the transient response lasted for around 100 seconds after which the sensor was saturated, and the sensor output was stable. Within these experiments, the thickness of the Ninhydrin-PDMS composite film was 250 μm. The saturated sensor output plotted against various concentrations of ammonia shows that, the sensor response is linear as shown in FIG. 8 yielding an output sensitivity of 0.0382 V/ppm.

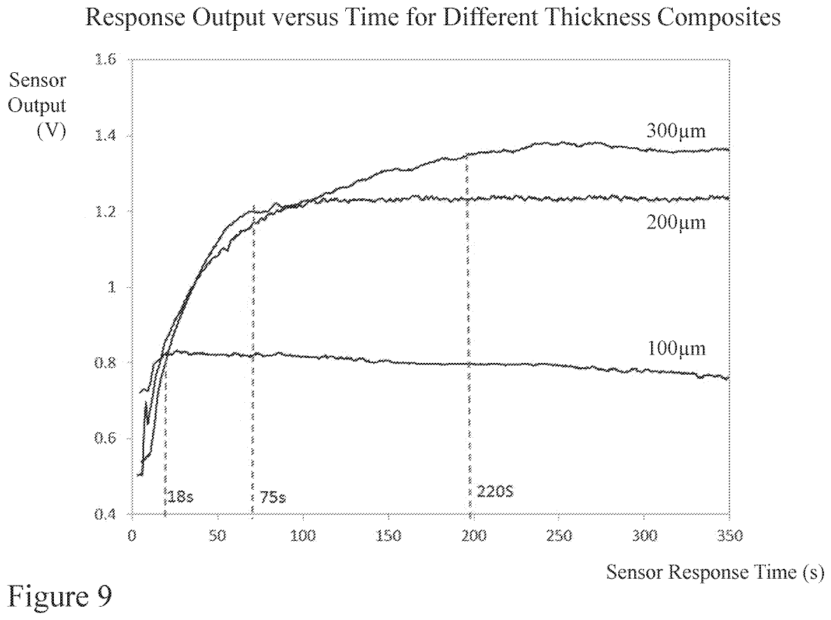

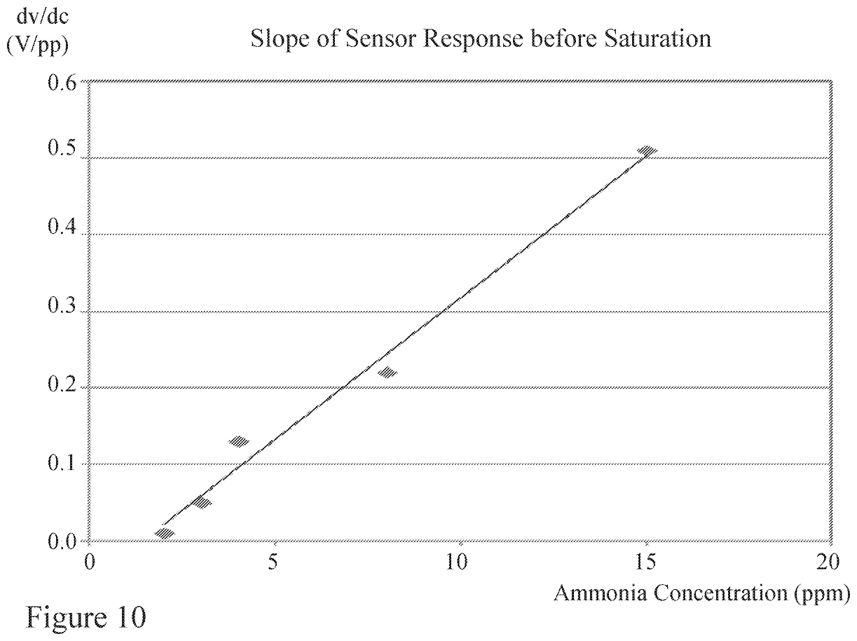

Subsequently, the inventors carried out tests with varying thickness of the Ninhydrin-PDMS composite film and found that the response time can be reduced to a lower value, by reducing the thickness of the film. The limit of detection in the present case was found to be as low as 2 ppm. In each case the composite was spun on a silicon wafer to obtain various thicknesses and the response time was investigated. FIG. 9 shows the response time of the Ninhydrin-PDMS composite film with thicknesses of 100 μm, 200 μm, and 300 μm respectively. The sensor response shows that a thinnest film response was the fastest (18 sec). It can also be observed from FIG. 7 that the absorbance of the Ninhydrin-PDMS composite film is slowly increasing linearly and after a few tens of seconds the absorbance becomes saturated. The slope of the linear response in the transient stage and the of saturation of the sensor output were found to be a function of the ammonia concentration. The effect of thickness of the film and the concentration of ammonia is also investigated as shown in FIG. 9 which shows that the slope of the transient response curve is approximately independent of thickness of the Ninhydrin-PDMS composite film, but the level of saturation varies with the thickness of the Ninhydrin-PDMS composite. From the experiments, it is shown that the sensor's response time for the Ninhydrin-PDMS composite film thicknesses investigated was between 20 seconds and 100 seconds. However, by using the slope of the response, the inventors not that the concentration of ammonia can be estimated within a shorter time frame, approximately between 5 seconds and 10 seconds and potentially faster, by measuring its slope as the film reacts with ammonia. This being evident from FIG. 10 where the slope of the sensor response against the concentration of ammonia is plotted.

### D: EXAMPLE OF ACTIVE MATERIAL COMPOSITE AMINO ACID SENSOR

Having demonstrated the ammonia sensing capabilities of the Ninhydrin-PDMS composite film the inventors proceeded to investigate the feasibility of detecting amino acids with the developed Ninhydrin-PDMS composite. In these experiments the inventors employed glycine as the amino acid being detected. As with the ammonia sensor the absorbance spectrum of the Ninhydrin-PDMS composite was measured when it reacted with glycine. As the reaction of the glycine with the Ninhydrin-PDMS composite is very slow at the room temperature the composite was annealed at 100° C. for 5 minutes. The heating process resulted in a faster reaction of composite with the glycine arid the color of the composite changed to slightly blue. Accordingly, thin film composites may be locally heated within the microfluidic device through thin film heaters, power resistors disposed adjacent, etc.

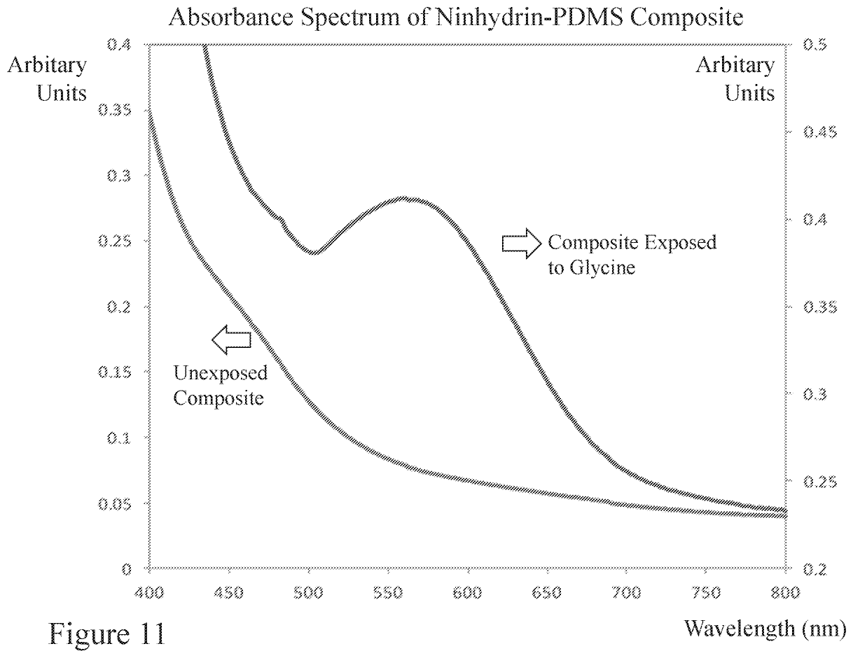

The resulting absorption peak as evident from FIG. 11 being within the region 540 nm-610 nm, namely the yellow region of the visible spectrum with some overlap down into the green and up into the orange. Accordingly, suitable laser or LED sources may include those exploiting AlGaInP or nitrogen doped GaP for the yellow-orange portion and AlGaInN or GaP for the green portion.

The embodiment of the invention may be employed to detect an analyte with an NxHy group.

Some active materials, e.g. ninhydrin, exhibit a shift of absorbance peak which varies with the amine group species absorbed. Accordingly, a microfluidic circuit employing a ninhydrin-PDMS composite in combination with a tunable wavelength optical system allows for the species-specific detection of any amine group by using spectroscopy. The tunable wavelength optical system may comprise a tunable laser with broadband optical detectors or a broadband optical source in combination with a tunable filter and broadband optical detector.

### E: ACTIVE MATERIAL COMPOSITE FILM ANALYSER

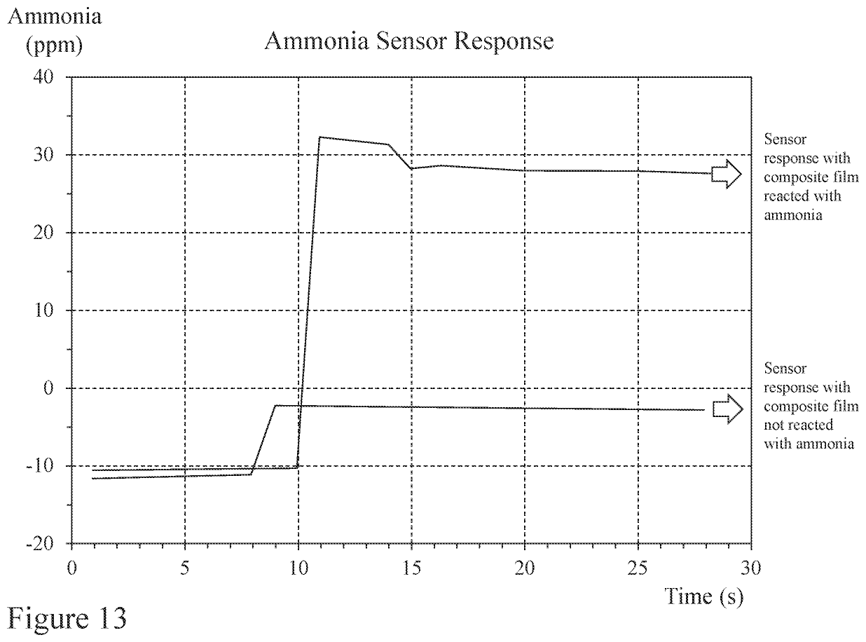

Referring to FIG. 12 there is depicted a packaged sensor with electronic hardware is shown in FIG. 12. In contrast to the device discussed and described supra in respect of FIGS. 5 and 6 the sensor packaging includes a slot to insert the Ninhydrin-PDMS composite film in the form of a strip. Accordingly, once Ninhydrin-PDMS composite has been exposed to ammonia the film is inserted into the specially designed slot of the device in order to assess the optical absorbance characteristics of film at the specific wavelength (450 nm), to perform the detection and quantification of ammonia. The sensor was interfaced with a microcontroller programmed to acquire data and perform the analysis. The resulting responses of the sensor device when films which have and have not been exposed to ammonia are inserted are depicted in FIG. 13. These results can be improved and are representative of an initial prototype device. For example, a sensor device with dual optical elements such that a single film element with base material (reference) or unreacted active material composite and active material composite (sample) would allow subtraction of the reference sample absorption.

Further, the microcontroller and associated electronics for power, power management, optical source/optical detector control, data acquisition and analysis may be implemented within an overall device with the microfluidic circuit. Accordingly, embodiments of the invention may include, but not be limited to:
Devices that receive exposed “film” (namely active material composite film) and perform measurements and/or analysis;
Devices containing “film” (namely active material composite film) or regions with the active material composite and perform measurements and/or analysis based upon a sample being loaded or acquired by the device through a loading process wherein the microfluidics circuit may or may not perform additional processing of the sample;
Devices containing “film” (namely active material composite film) or regions with the active material composite and perform measurements and/or analysis based upon a sample being periodically or continuously acquired by the device (e.g. fluid flow from an inlet to outlet) wherein the microfluidics circuit may or may not perform additional processing of the sample;
Devices may support singe use, continuous use, and multiple uses (e.g. multiple sensors with fluidic switching circuit so that after a sensor is used the fluidic switching circuit reconfigures to enable another sensor); and
Devices may employ sensing elements exploiting active material composites that undergo a reversible transition process when exposed to the analyte or undergo a permanent transition process when exposed to the analytes.

### F: INITIAL EMBODIMENTS

Referring to FIGS. 3 and 13 together with the descriptions above initial prototype devices according to embodiments of the invention have been described and depicted. Accordingly, these initial prototype devices without significant optimization have been demonstrated to provide measurements over the range 1 ppm to 1,000s ppm and with a sensitivity that is scalable. As described below arrayed configurations may be implemented with multiple analytes and/or reagents but these may also be employed to provide different sensors with different ranges within the same device, dependent upon the application(s).

Embodiments of the invention may also be designed and implemented to provide sensitivities in the parts per billion (ppb) range. Further, initial embodiments of the invention have been operated over initial temperature ranges of −4° C. to +100° C. with responses times of seconds over this temperature range. These initial embodiments have also been demonstrated to be independent of humidity effects through experiments performed with water based ammonia solutions and ammonia vapour alone.

### G: ALTERNATE EMBODIMENTS AND EXTENSIONS OF ACTIVE MATERIAL SENSOR CONCEPT

G1: Electronics and Reference Elements

Within the prototype devices exploiting an embodiment as discussed and described with respect to FIGS. 1 to 13 respectively the optical signal from the optical source, e.g. LED or laser diode, is transmitted through the active material composite and received by an optical detector wherein the output voltage is monitored, and the analyte level determined in dependence of the optical detector output. As noted supra any inherent absorption within the active material composite may be referenced out by providing a reference element not exposed to the analyte. This may even exploit a common optical source and a differential amplifier coupled to a pair of optical detectors such that only the difference is amplified for signal processing and analysis.

Optionally, the front-end amplifier may be replaced with an instrumentation amplifier or a fully-differential instrumentation amplifier. Optionally, the front-end electronics may provide a lock-in amplifier function either with analog signal processing or digital signal processing wherein depending upon the dynamic range of the electronics signals up to 10⁶ times smaller than noise components within the received signal can be detected.

Accordingly, embodiments of the invention may support detection at parts per billion (ppb) levels of the analyte.

G2: Microfluidic Circuit Design

Within the initial prototype described and depicted supra in respect of FIGS. 5, 6 and 12 respectively the optical path through the active material composite is a single pass. However, it would evident that within other embodiments of the invention the microfluidics and active material composite may be implemented such that the optical path traverses either multiple passes through a single active material composite film or multiple active material composite elements. According, examples of such alternate microfluidic circuits exploiting embodiments of the invention are depicted in FIGS. 14 to 17 respectively.

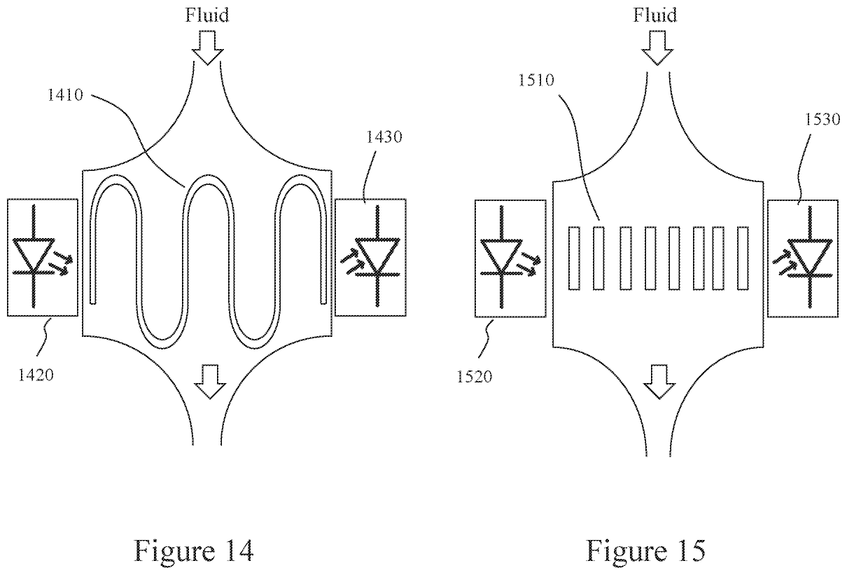

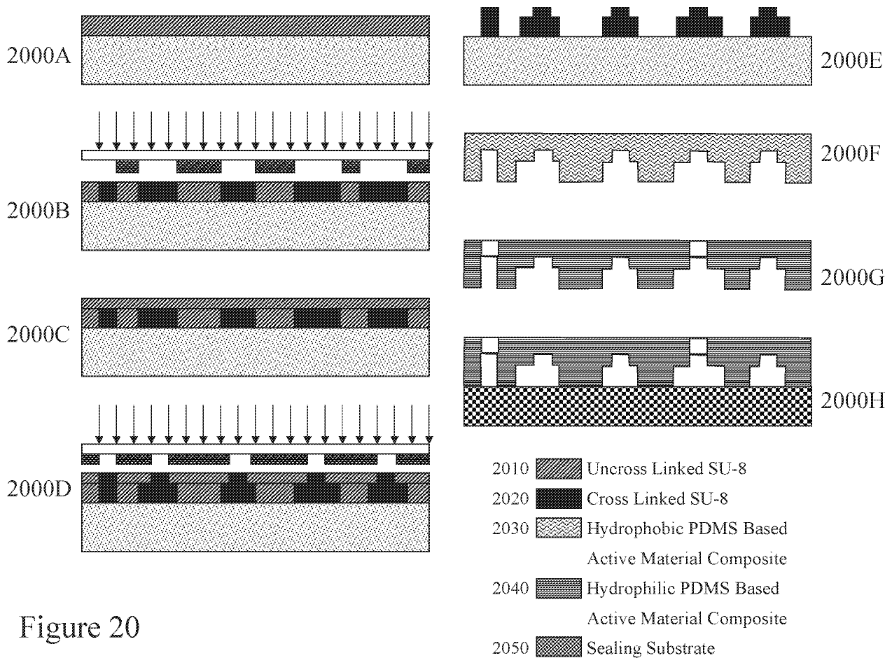

Referring to FIG. 14 an active matrix composite film 1410 is depicted shaped into a meandering structure in the fluid flow from inlet to outlet. Accordingly, the fluid engages a greater surface of the active matrix composite and the optical path between the optical source 1420 and optical detector 1430 traverses multiple sections of the active matrix composite. In contrast, in FIG. 15, the active matrix composite regions 1510 represent a series of isolated regions within the overall fluidic channel from inlet to outlet. Accordingly, in common with FIG. 14 the fluid engages a greater surface of the active matrix composite and the optical path between the optical source 1420 and optical detector 1430 traverses multiple sections of the active matrix composite but with reduced resistance to the fluid flow. Accordingly, as discussed below in respect of FIG. 20 these regions of active matrix composite may be formed through a variety of processes concurrent with or subsequent to the formation of the microfluidic device. Optionally, other geometries of the active matrix composite regions 1510 may be employed within the boundaries of the particular manufacturing process(es) employed for their formation. Optionally, in its simplest form a sensor according to an embodiment may be a microfluidic channel within a structure wherein the entire microfluidic channel is formed from the active matrix composite and the optical probe is transmitted through part or all of the microfluidic channel. For example, an active matrix composite with a channel or hole through it through which the analyte flows illuminated on one side with an appropriate optical source and on the other by appropriate optical detector may establish the increased absorption as discussed with respect to a Ninhydrin-PDMS composite and variants thereof.

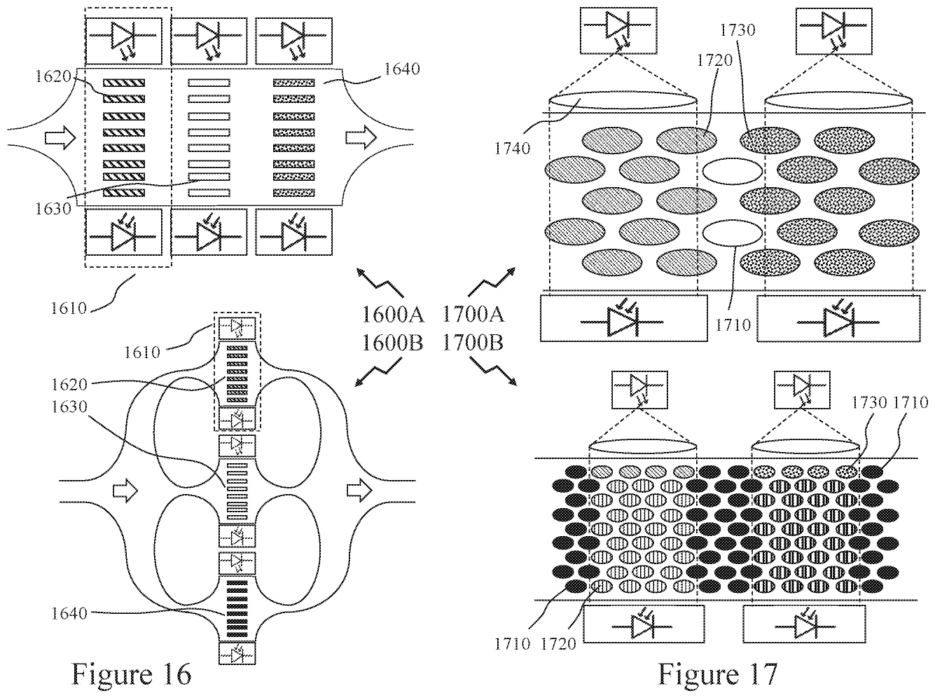

Now referring to FIG. 16 multi-analyte embodiments are depicted in first and second schematics 1600A and 1600B respectively. Within the microfluidic circuit in first image 1600A first to third active matrix composite regions 1620 to 1640 are depicted sequentially along the microfluidic device wherein each of the first to third active matrix composite regions 1620 to 1640 is disposed between an optical circuit 1610 comprising an optical source and optical detector. Accordingly, in dependence upon the absorbance spectrum of each of the first to third active matrix composite regions 1620 to 1640 with respect to their analyte the optical operating parameters of each optical source and optical detector are determined. Accordingly, for a multi-analyte sensor each of the first to third active matrix composite regions 1620 to 1640 may employ a different active material within the respective active matrix composite. Second image 1600B depicts a similar multi-analyte microfluidic sensor with first to third active matrix composite regions 1620 to 1640 but these are now disposed in parallel with respect to the flow through the microfluidic device.

Now referring to FIG. 17 multi-analyte embodiments are depicted in first and second schematics 1700A and 1700B respectively wherein the active matrix composite based sensor is now integrated within a microfluidic circuit element, namely a low pressure capillary pump in first image 1700A and a high pressure capillary pump in second image 1700B. Accordingly, such capillary pumps may draw samples, processed samples etc. through a microfluidic circuit, such as a lab-on-a-chip, and integrate sensors within the capillary pumps. In each of the first and second images 1700A and 1700B respectively the capillary pump comprises a plurality of regions with elements of predetermined shape and microfluidic channels between them. Accordingly, there are base polymer elements 1710 wherein the polymeric material of the microfluidic device is employed, first active material elements 1720 wherein a first active material composite is employed, and second active material elements 1730 wherein a second active material composite is employed. Those regions with first and second active material elements 1720 and 1730 being disposed between optical circuits to illuminate and detect. In addition to the optical source and optical detector the optical circuits may incorporate a lens 1740 to reduce the divergence of the optical source, such as a LED or laser diode, allowing reduced crosstalk between adjacent active material composite sensors or operation at lower optical power. Optionally, another lens may be employed before the optical detector to enable use of lower area photodetectors in conjunction with larger area active material composites.

Optionally, the active material composite may be employed within other microfluidic circuit elements including, but not limited to, symmetric capillary pumps; asymmetric programmable capillary pumps; microchannels; serpentine flow resistors; capillary pumps; reservoirs; and flow routers. Accordingly, active material composite sensors may be employed at different points within a microfluidic circuit.

G3: Nano-Structured and Micro-Structured Composites

Within the embodiments described and depicted supra in respect of FIGS. 1 to 17 the active material composites and sensors exploiting active material composites have been described as exploiting an active material within a polymer structure. As described the structures are assumed to be solid such as formed by spinning, depositing, and/or coating a liquid medium comprising the polymer and active material onto a substrate such as a substrate formed from the polymer although other substrates may be employed including but not limited to silicon, another polymer, an elastomer, a glass, a ceramic, etc. provided that in addition to supporting fluid flow through microfluidic structures the optical probe signal from the optical source can be coupled to the active material composite and the resulting transmitted signal detected such that the analyte's presence and concentration, volume, etc. determined.

However, within other embodiments of the invention the active material composite may include microparticles and/or nanoparticles (hereinafter referred to as microparticles) in order to adjust the optical properties of the active material composite or to provide additional aspects such as increased interfaces, mechanical integrity, etc. Optionally, these microparticles may be also embedded into other regions of the microfluidic circuit to adjust mechanical integrity, adjust optical properties etc. Microparticles may be metallic, dielectric, semiconductor, magnetic, non-magnetic, ceramic, polymeric, etc. according to the target characteristics of the material and not impeding the sensing functionality of the active material where they are deployed within the active material composite. The microparticles may be solid, semi-solid, or soft. For example, clay microparticles into polymer matrices increase reinforcement, leading to stronger plastics, verifiable by a higher glass transition temperature and other mechanical property tests. Zinc oxide microparticles can provide UV blocking properties.

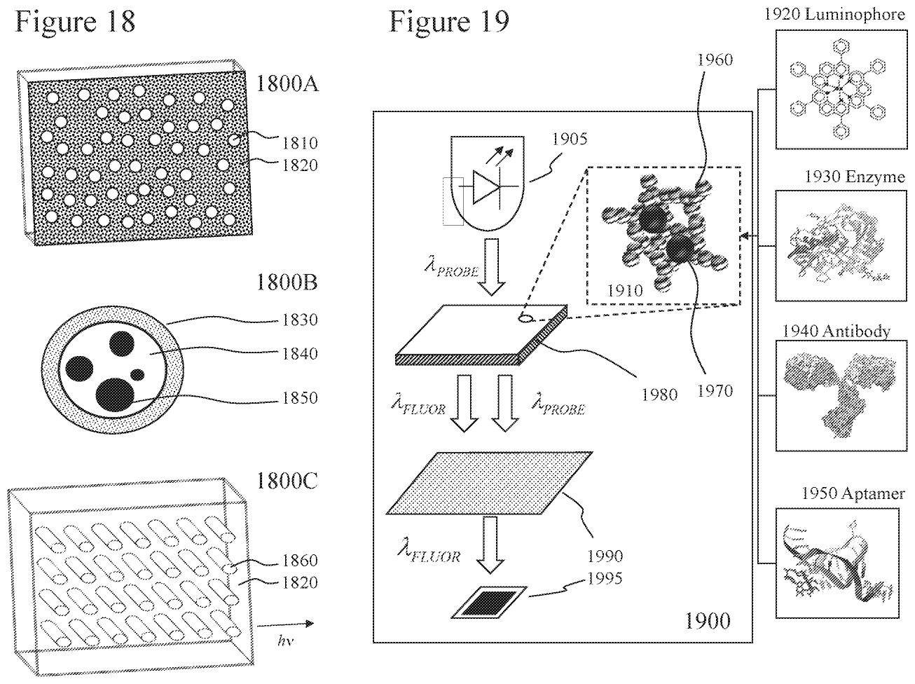

Accordingly, referring to FIG. 18 in first image 1800A the active material composite 1820 has embedded within it microparticles 1810 which may for example be solid, hollow, or may be structured. Alternatively, the microparticles 1810 may be porous, e.g. mesoporous silica. Referring to second image 1800B an exemplary schematic of a structured microparticle is depicted comprising a plurality of nanoparticles 1850 within a body 1840 with or without an additional coating 1830. Optionally, the microparticles may be microparticles with solid core, microparticles with non-solid core, microparticles with solid microdomains or nanodomains within a matrix, microparticles with non-solid microdomains or nanodomains within a matrix, or a mixture of an encapsulated agent within a matrix. Optionally, the microparticles may contain different active material to the active material within the active material composite they are disposed within. Within third image 1800C the active material composite 1820 is depicted with an array of nanostructures 1860 embedded within in a regular lattice formation. Optionally, the nanostructures 1860 may be irregularly disposed. Optionally, the nanostructures 1860 may be nanotubes formed from a material such as carbon nanotubes. Optionally, the nanostructures 1860 may be aligned in a different direction relative to the optical source-optical detector path than the one depicted in third image 1800C.

G4: Fluorescent Based Active Material Composite Sensors

Within the embodiments described and depicted supra in respect of FIGS. 1 to 17 the active material composites and sensors exploiting active material composites exploit absorption by the active material of an optical signal to provide an optical interrogation of the sensor. These embodiments have been described with respect to transmission through the active material composite from optical source to optical detector disposed on opposite sides of the active material composite although reflectance from an active material composite from optical source to optical detector on the same side of the active material composite may be applicable in respect of other active materials and their associated analytes.

However, within other embodiments an active material composite may be employed exploiting fluorescence with the active material being a fluorescent sensor material. Such a fluorescence based system 1900 may comprises a fluorescent material 1910 embedded within a polymer material 1980. The fluorescent material 1910 as depicted comprising a capture material 1960 and a fluorescent material 1970. According to the analyte being sensed capture material may be a luminophore 1920, enzyme 1930, antibody 1940 or aptamer 1950. In the instance that the capture material is itself a luminophore 1920 and hence luminescent itself the fluorescent material 1970 may be omitted. Although in other instances wherein the luminophore 1920 is more akin to a phosphor a fluorescent material 1970 may be employed to shift the detection wavelength to a region away from the optical probe wavelength exciting the measurement system. As depicted in FIG. 19 with schematic 1900 the capture material 1960 and a fluorescent material 1970 are embedded within the composite 1980. An optical signal from an optical source 1905 at a probe wavelength, λ_{PROBE}, wherein fluorescent emission occurs if the analyte has bound to the fluorescent material 1970 at a different wavelength, λ_{FLUOR}. Accordingly, if λ_{PROBE} is filtered out by filter 1990 then only the optical signal at λ_{FLUOR} impinges onto the optical detector. Accordingly, the material selection for the composite must now consider two different wavelengths, λ_{PROBE} and λ_{FLUOR} wherein λ_{PROBE} is typically in the blue ultraviolet regions of the electromagnetic spectrum and λ_{FLUOR} is longer than λ_{PROBE} and typically in the visible or near infra-red.

Considering, luminophore 1920 then this can be divided into two subcategories, fluorophores and phosphors. The difference between luminophores belonging to these two subcategories is derived from the nature of the excited state responsible for the emission of photons. Some luminophores, however, cannot be classified as being exclusively fluorophores or phosphors and exist in the gray area in between. Such cases include transition metal complexes whose luminescence comes from an excited (nominally triplet) metal-to-ligand charge transfer (MLCT) state, but which is not a true triplet-state in the strict sense of the definition; and colloidal quantum dots, whose emissive state does not have either a purely singlet or triplet spin. Most luminophores consist of conjugated pi systems or transition metal complexes. In addition, purely inorganic luminophores, such as zinc sulfide doped with rare earth metal ions, rare earth metal oxysulfides doped with other rare earth metal ions, yttrium oxide doped with rare earth metal ions, zinc orthosilicate doped with manganese ions, etc.

Enzymes 1930, like catalysts, work by lowering the activation energy for a reaction, thus dramatically increasing the rate of the reaction. Enzymes are very selective and speed up only a few reactions, which given that enzymes are known to catalyze about 4,000 biochemical reactions, implies the number of potential enzymes available is large. A few RNA molecules called ribozymes also catalyze reactions, with an important example being some parts of the ribosome. Additionally, synthetic molecules called artificial enzymes also display enzyme-like catalysis adding to the pool of available capture molecules to operate in conjunction with the fluorescent material to establish optical activity in dependence of the process they are monitoring, controlling, or accelerating.

An antibody 1940, also known as an immunoglobulin, is a large Y-shaped protein used by the immune system to identify and neutralize foreign objects such as bacteria and viruses. The antibody recognizes a unique part of the foreign target, termed an antigen. Though the general structure of all antibodies is very similar, the small region at the tip of the protein is extremely variable, allowing millions of antibodies with slightly different tip structures, or antigen binding sites, to exist. This region is known as the hypervariable region. Each of these variants can bind to a different target. Accordingly, there is enormous diversity in the antibodies which can be exploited.

Aptamers 1950 are oligonucleic acid or peptide molecules that bind to a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. More specifically, aptamers can be classified as either DNA or RNA aptamers in that they consist of (usually short) strands of oligonucleotides or peptide aptamers in that they consist of short variable peptide domains, attached at both ends to a protein scaffold.

Accordingly, a wide range of materials can be employed as capture material and bound within a matrix allowing fluorescent based optical sensors to be integrated with a compact footprint and low cost. Whilst generally different capture materials would be employed for different analytes multiple capture materials may also be employed for a single analyte, such as for example to provide an increased dynamic range of measurement than is achievable with a single capture material.

### H: MICRO-FLUIDIC AND NANO-FLUIDIC CIRCUIT MANUFACTURING

Referring to FIG. 20 an exemplary process flow for the fabrication of a microfluidic circuit exploiting an active material composite. Within this embodiment SU-8 is employed as a mold for the fabrication microstructures or nanostructures of active material composite.

In first step 2000A an uncrosslinked SU-8 layer 2010 is deposited upon a substrate. Next in step 2000B this uncrosslinked SU-8 layer 2010 is exposed through optical lithography defining crosslinked SU-8 regions 2020. This process is repeated in steps 2000C and 2000D to provide two layers of cross-linked SU-8. SU-8 being an epoxy-based negative photoresist that is very viscous polymer and can be spun over thicknesses ranging from up to and still be processed with standard contact lithography. The uncrosslinked SU-8 material is removed in step 2000E wherein the resulting crosslinked SU-8 2020 forms the basis for a molding of hydrophobic PDMS based active material composite 2030 which may be spun or poured to form the molded element. Next in step 2000F the hydrophobic PDMS based active material composite 2030 is processed to provide hydrophilic PDMS based active material composite 2040 and vent holes are formed. Next in step 2000G a sealing substrate 2050 is attached to form the microfluidic circuit. Optionally, vent holes may be implemented within the sealing substrate 2050 or the PDMS based active material composite. Optionally, sample loading points, inlet ports, and outlet ports may be provided within the PDMS based active material composite and/or sealing substrate 2050. Within embodiments the sealing substrate 2050 may me a PDMS substrate formed with features via a similar process as that depicted within FIG. 20 or it may also be a PDMS based active material composite. As noted supra in Section B1 other materials other than PDMS may employed as the host material within the active material composite.

The photolithographic patterning process described and depicted in respect of FIG. 20 may be employed to form microfluidic devices as described and depicted in respect of FIGS. 3, 5 and 14-17 with regions formed from one or more active material composites within a microfluidic device formed from an inert or passive material. Optionally, the photolithography, deposition, lift-off process may be employed to form the regions with active material composite and then repeated for each other active material composite employed within the microfluidic circuit.

Optionally, a photolithography, deposition and etching process may be employed to removed cured active material composite or solid active material composite. Optionally, photolithography, deposition and etching processes may be employed to structure passive or inert materials to form the upper and/or lower portions of the microfluidic circuit within materials such as silicon, silica, fused quartz, alumina, glass etc. onto which the one or more regions of active material composite are formed.

Optionally, other manufacturing processes within the semiconductor processing industry may be employed to form, pattern the active material composite and/or passive materials. Optionally, the active material composite may be stamped, molded, etched, machined, etc. according to the manufacturing process or processes employed. Stamping may, for example, employ LIGA techniques to create high-aspect-ratio stamps for the microstructures.

The foregoing disclosure of the exemplary embodiments has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

1. A device comprising:
a sensor;
an inlet port for coupling a fluid to the sensor; and
an outlet port for coupling fluid from the sensor.

2. The device according to claim 1, wherein
the sensor comprises an active material composite comprising a passive material and an active material dispersed within the passive material; wherein
the active material is selected in dependence upon a target analyte to be measured; and
at least one of:
the active material composite further comprises a polymer additive comprising at least one an electrically conductive polymer additive and an electrically non-conductive polymer additive; and
the active material composite further comprises at least one of microparticles and nanoparticles wherein the microparticles are either solid, hollow, or incorporate a plurality of nanoparticles within a matrix.

3-4. (canceled)

5. The device according to claim 1, wherein
the sensor comprises an active material composite within a predetermined portion of the device, the active material composite comprising a passive material, an active material supporting an interaction with an analyte and a filler; wherein
either:
the filler comprises at least one of metallic nanoparticles and electrically conductive nanoparticles wherein an interaction of at least one of a biological analyte and a chemical analyte with the active material is interrogated by electrical impedance spectroscopy;
or
the filler comprises at least one of electrically conducting nanoparticles and electrically non-conducting nanoparticles incorporated with multiple layers of the active material composite to enhance the sensitivity of detecting the analyte interaction by increasing the surface area of first active material exposed to the analyte.

6. The device according to claim 1, wherein
the sensor comprises an active material composite comprising a passive material and an active material dispersed within the passive material; wherein
the active material is selected in dependence upon a target analyte to be measured.

7. The device according to claim 1, wherein
the sensor comprises a first portion forming part of a microfluidic circuit and a second portion either integrated as part of the first portion or removably insertable into the first portion;
the second portion of the sensor comprises an active material composite comprising a passive material and an active material dispersed within the passive material;
the active material is selected in dependence upon a target analyte to be measured; and
at least one of:
the passive material comprises at least one of a substantially non-electrically conductive base elastomer and a substantially non-electrically conductive polymer; and
the active material composite further comprises a particulate filler comprising at least one of electrically non-conductive particles and electrically conductive particles.

8. The device according to claim 1, wherein
the sensor comprises an active material composite comprising a passive material and an active material dispersed within the passive material; wherein
the active material composite comprises at least one of an elastomer and polymer in combination with a plurality of active materials of which the active material is one;
the plurality of active materials are employed at least one of together within a region of the predetermined portion of the device, within multiple regions of the predetermined portion of the device with a different active material in each region, and within multiple regions of the predetermined portion of the device with varying subsets of the plurality of active materials in each region; and
the plurality of active materials are selected in dependence upon one or more analytes to be analysed.

9. (canceled)

10. The device according to claim 1, wherein
the sensor comprises a first portion forming part of a microfluidic circuit and a second portion either integrated as part of the first portion or removably insertable into the first portion; and
the active material is selected in dependence upon a target analyte to be measured.

11. The device according to claim 10, wherein
the active material is a reagent; and
the reagent exhibits a change in at least one of an optical characteristic and an electrical characteristic in dependence upon an amount of the target analyte the sensor is exposed to.

12. The device according to claim 10, wherein
the active material forms a predetermined portion of a microfluidic structure within the predetermined portion of the device.

13. The device according to claim 10, wherein
the active material is ninhydrin;
the target analyte is at least one of ammonia, an amino acid and an NxHy group; and
the passive material is one of:
polydimethylsiloxane (PMDS);
an optically transparent polymer; and
selected from the group comprising poly(methyl methacrylate) (PMMA), SU8, a Cyclic Olefin Copolymer (COC), a Cyclic Olefin Polymer (COP), and a COC/COP mixture.

14-16. (canceled)

17. The device according to claim 10, further comprising
an optical source emitting optical signals over a first predetermined wavelength range established in dependence upon the optical properties of the active material;
an optical detector detecting optical signal over a second predetermined wavelength range established in dependence upon the optical properties of the active material; wherein
if the optical properties of the active material are a change in absorption a predetermined portion of the second predetermined wavelength range overlaps a predetermined portion of the first predetermined wavelength range; and
if the optical properties of the active material are fluorescence then the second predetermined wavelength range does not overlap the first predetermined wavelength range.

18. A microfluidic circuit comprising:
a microfluidic element forming a portion of the microfluidic circuit; wherein
a predetermined portion of the microfluidic element is formed from an active material composite comprising a passive material and an active reagent dispersed within the passive material.

19. The microfluidic circuit according to claim 18, wherein
the active material is a reagent; and
the reagent exhibits a change in at least one of an optical characteristic and an electrical characteristic.

20. The microfluidic circuit according to claim 18, wherein
the active material is ninhydrin;
the target analyte is at least one of ammonia, an amino acid and an NxHy group;
the active material exhibits a change in optical absorption within a predetermined wavelength range; and
the passive material is one of:
polydimethylsiloxane (PMDS);
an optically transparent polymer; and
selected from the group comprising poly(methyl methacrylate) (PMMA), SU8, a Cyclic Olefin Copolymer (COC), a Cyclic Olefin Polymer (COP), and a COC/COP mixture.

21. (canceled)

22. The microfluidic circuit according to claim 18, further comprising
an optical source emitting optical signals over a first predetermined wavelength range established in dependence upon the optical properties of the active material;
an optical detector detecting optical signal over a second predetermined wavelength range established in dependence upon the optical properties of the active material; wherein
if the optical properties of the active material are a change in absorption a predetermined portion of the second predetermined wavelength range overlapping a predetermined portion of the first predetermined wavelength range; and
if the optical properties of the active material are fluorescence then the second predetermined wavelength range does not overlap the first predetermined wavelength range.

23. The microfluidic circuit according to claim 18, wherein
the microfluidic element is one of at least one of a symmetric capillary pump; an asymmetric programmable capillary pump, a microchannel, a reservoir, a serpentine flow resistor, a capillary pump and a flow router; and
the predetermined portion of the microfluidic element is at least one of a sidewall of the microfluidic element, a lower surface of the microfluidic element, an upper surface of the microfluidic element and an element disposed within the microfluidic element.

24-31. (canceled)

32. A method comprising:
fabricating a first predetermined portion of a microfluidic circuit from a predetermined material; and
fabricating a second predetermined portion of the microfluidic circuit from a composite material; wherein
the composite material comprises a passive material and an active reagent; and
the active reagent exhibits either a change in optical absorption within a predetermined optical wavelength range upon exposure to a predetermined analyte or fluorescence upon exposure to the predetermined analyte; and
the predetermined material is optically transparent within the predetermined optical wavelength range allowing monitoring of the change in either the optical absorption or fluorescence through the first predetermined portion of the microfluidic circuit and the second predetermined portion of the microfluidic circuit.

33. The method according to claim 32, wherein
fabricating the second predetermined portion of the microfluidic circuit from the composite material comprises:
dissolving ninhydrin within a suitable solvent to form a ninhydrin mixture;
mixing polydimethylsiloxane (PDMS) with a curing agent and base material to form a PDMS mixture;
combining and mixing the ninhydrin mixture and the PDMS mixture;
spin coating the ninhydrin-PDMS mixture to form a thin film; and
processing the thin film to form the composite material and define the second predetermined portion of the microfluidic circuit.

34-35. (canceled)

36. The method according to claim 32; wherein
fabricating the second predetermined portion of the microfluidic circuit from a composite material comprises:
providing a passive structural material;
providing an active material exhibiting a variation in a property in dependence upon exposure to the predetermined analyte; wherein
forming a single composite material by mixing the active material and the passive structural material; and
fabricating the second predetermined portion of the microfluidic circuit comprises forming either:
a thin film of the single composite material for removable insertion into the first predetermined portion of the microfluidic circuit as part of a sensor for the predetermined analyte; or
as a first monolithically integrated portion of the first predetermined portion of the microfluidic circuit to provide a sensor for the predetermined analyte in conjunction with a second monolithically integrated portion of the sensor from a predetermined material.

37. The method according to claim 32, wherein
at least one of:
the time dependent response of either the optical absorption or fluorescence is monitored and a slope of the time dependent response of either the optical absorption or fluorescence is employed to determine the concentration of the analyte;
the second predetermined portion of the microfluidic circuit is a removable element comprising at least a film;
the second predetermined portion of the microfluidic circuit comprises is a removable element comprising at least a film having a first region comprising the composite material such that the composite material can be exposed to the predetermined analyte and a second region comprising the composite material but preventing exposure of the composite region to the predetermined analyte;
the second predetermined portion of the microfluidic circuit comprises is a removable element comprising at least a film having a first region comprising the composite material and a second region comprising the passive material.
